(19) United States
(12) Patent Application Publication
(10) Pub. No.: US 2025/0151218 A1
(43) Pub. Date: May 8, 2025

(54) TILTABLE DISPLAY SCREEN ASSEMBLIES, DIAGNOSTIC ANALYZERS WITH TILTABLE DISPLAY SCREEN ASSEMBLY, AND METHODS

(71) Applicant: Siemens Healthcare Diagnostics Inc., Tarrytown, NY (US)

(72) Inventor: William Chura, Newton, MA (US)

(73) Assignee: Siemens Healthcare Diagnostics Inc., Tarrytown, NY (US)

(21) Appl. No.: 18/730,519

(22) PCT Filed: Jan. 20, 2023
(86) PCT No.: PCT/US2023/060945

§ 371 Date: Jul. 19, 2024

Related U.S. Application Data
(60) Provisional application No. 63/267,205, filed on Jan. 27, 2022.

(51) Int. Cl. H05K 5/02; F16M 11/10

(52) U.S. Cl. CPC H05K 5/0234; F16M 11/10; F16M 2200/024

(57) ABSTRACT

A tiltable display screen assembly of a diagnostic analyzer. The tiltable display screen assembly includes a tiltable support interconnectable to a hand-held device including a display screen, a multi-angle adjuster having adjustment features, and a tilting screen mechanism pivotable relative to the tiltable support. The tilting screen mechanism includes a shaft, one or more moveable paddles coupled to the shaft, an adjustment member depending from the shaft configured to engage with the adjustment features of the multi-angle adjuster in order to adjust a tilt angle of the tiltable support and the display screen of the hand-held device. Diagnostic analyzers including the tiltable display screen assembly and methods for adjusting tilt angle of a display screen of a diagnostic analyzer are described, as are other aspects.

This application claims benefit under 35 USC § 119(e) of U.S. Provisional Application No. 63/267,205, filed Jan. 27, 2022. The entire contents of the above-referenced patent application are hereby expressly incorporated herein by reference.

### FIELD

This disclosure relates to assemblies that provide for tilting of a display screen, diagnostic analyzers including tiltable display screen assemblies, and methods thereof.

### BACKGROUND

In some medical diagnostics, such as point-of-care medical diagnostics, a sample containing biological material or a component thereof can be inserted into the inner confines of a diagnostic instrument or device (hereinafter “diagnostic analyzer”). Such diagnostic analyzers include, but are not limited to, immunoassay instruments adapted to perform molecular tests, hematology analyzers, blood gas analyzers, and chemical analyzers that analyze samples as to their chemical makeup and/or to determine an analyte thereof. The sample may be a raw sample of, or containing, biological material, such as whole blood, blood serum or plasma, plural fluid, urine, interstitial fluid, cerebrospinal fluid, or the like, or a biological sample that may have been previously processed so as to extract a protein, analyte, or constituent of interest to be identified, characterized, and/or measured.

Improvements to the functionality of such diagnostic analyzers and methods are needed that enable improved operation and utilization thereof.

### SUMMARY

In one embodiment, a tiltable display screen assembly is provided. The tiltable display screen assembly comprises a tiltable support configured to be interconnectable to a hand-held device comprising a display screen, a multi-angle adjuster having multiple adjustment features, and a tilting screen mechanism pivotable relative to the tiltable support, the tilting screen mechanism comprising: a shaft, one or more moveable paddles coupled to the shaft, and an adjustment member extending from the shaft and having an end configured to engage with two or more of the multiple adjustment features of the multi-angle adjuster in order to adjust a tilt angle of the tiltable support.

In another embodiment, a diagnostic analyzer is provided. The diagnostic analyzer comprises a base, a hand-held device rotatably coupled to the base, the hand-held device comprising a display screen, and a tiltable display screen assembly interconnected between the base and the hand-held device. The tiltable display screen assembly comprises a tiltable support interconnected to the hand-held device, the tiltable support comprising a support bracket attached to a mounting member that is configured to attach to the hand-held device, a multi-angle adjuster coupled to the base, the multi-angle adjuster having multiple adjustment features thereon, and a tilting screen mechanism pivotable relative to the tiltable support. The tilting screen mechanism comprises a shaft, one or more moveable paddles coupled to the shaft, and an adjustment member extending from the shaft and having an end configured to engage with two or more of the multiple adjustment features of the multi-angle adjuster in order to adjust a tilt of the display screen relative to the base.

In another embodiment, a method of positioning a display screen of a diagnostic analyzer is provided. The method comprises providing a base of the diagnostic analyzer, providing a hand-held device comprising the display screen, interconnecting a tiltable display screen assembly between the base and the hand-held device, wherein the tiltable display screen assembly comprises a tiltable support comprising a support bracket attached to a mounting member that is attached to the hand-held device, a multi-angle adjuster coupled to the base, the multi-angle adjuster having multiple adjustment features thereon, a tilting screen mechanism pivotable relative to the tiltable support, the tilting screen mechanism comprising: a shaft, one or more moveable paddles coupled to the shaft, and an adjustment member extending from the shaft and having an end configured to engage with at least some of the multiple adjustment features; and depressing the one or more moveable paddles to disengage the end of the adjustment member from one of the multiple adjustment features in order to tilt the tiltable support relative to the base and adjust a tilt angle (TA) of the display screen.

Still other aspects, features, and advantages of this disclosure may be readily apparent from the following description and illustration of a number of example embodiments, including the best mode contemplated for carrying out the disclosure. This disclosure may also be capable of other and different embodiments, and its several details may be modified in various respects, all without departing from the scope of the disclosure. This disclosure is intended to cover all modifications, equivalents, and alternatives falling within the scope of the claims.

### BRIEF DESCRIPTION OF THE DRAWINGS

The drawings, described below, are for illustrative purposes, and are not necessarily drawn to scale. Accordingly, the drawings and descriptions thereof are to be regarded as illustrative in nature, and not as restrictive. The drawings are not intended to limit the scope of the disclosure in any way.

FIG. 1A illustrates an isometric view of a first example embodiment of a tiltable display screen assembly according to the disclosure.

FIG. 1B illustrates another isometric view of the first example embodiment of tiltable display screen assembly according to the disclosure.

FIG. 1C illustrates partial side view of an example embodiment of a diagnostic analyzer including a tiltable display screen assembly with a tilting screen mechanism shown in a coupled configuration according to the disclosure.

FIG. 1D illustrates partial side view of an example embodiment of a diagnostic analyzer including a tiltable display screen assembly with a tilting screen mechanism shown in an uncoupled configuration wherein an adjustment member is shown disengaged from adjustment features of a multi-angle adjuster so that a tilt angle can be adjusted according to the disclosure.

FIG. 1E illustrates an isometric view of a tiltable display screen assembly including a mounting member according to the disclosure.

FIG. 1F illustrates a front isometric view of a hand-held device including a display screen configured to be mounted onto a tiltable display screen assembly according to the disclosure.

FIG. 1G illustrates a back isometric view of a hand-held device including mounting features configured to couple to a tiltable display screen assembly according to the disclosure.

FIG. 1H illustrates a partial side cross-sectional view of a hand-held device mounted to a mounting member and further illustrating a latch and catch mechanism configured to accomplish coupling of the hand-held device to the mounting member according to the disclosure.

FIG. 1I illustrates a partial isometric view of a bottom portion of a mounting member and coupled support bracket according to the disclosure.

FIG. 2A illustrates a side view of an example embodiment of a diagnostic analyzer including a tiltable display screen assembly shown in a raised configuration according to the disclosure.

FIG. 2B illustrates a side view of the example embodiment of a diagnostic analyzer including a tiltable display screen assembly shown in a lowered configuration to enable clearing of a cabinet and/or cabinet door according to the disclosure.

FIG. 3 illustrates a method of positioning a display screen of a diagnostic analyzer according to the disclosure.

### DETAILED DESCRIPTION

This disclosure is directed at tiltable display screen assemblies and methods that enable a display screen, such as a display screen of a diagnostic analyzer, to tilt. The disclosure is further directed to diagnostic analyzers comprising a tiltable display screen assembly. According to a first embodiment of the disclosure, a tiltable display screen assembly is provided that is configured to be interconnected to a hand-held device in a manner so that it is readily attachable and detachable therefrom and further so that the display screen thereof can be tilted to provide a changed or improved viewing angle and/or to lower the display screen height so that the display screen can be slid underneath of a storage cabinet or otherwise avoid interference with an operation of a component, such as a cabinet door.

In more detail, the tiltable display screen assembly, which can be a functional part of a diagnostic analyzer, such as a point-of-care diagnostic analyzer, allows for ease of tilt angle adjustment of a display screen thereof. In particular, medical personnel can readily change a tilt angle of the tiltable display screen assembly to which the hand-held device and display screen thereof is interconnected.

As used herein, the phrase “coupled to” means attached to (e.g., either fixedly or releasably) by any suitable attachment mechanism (e.g., screws, snap fit, press fit, or other mechanical attachment device, integral with, molded as part of, extending from, or formed as a subpart thereof. As used herein, “configured to be interconnectable to” means that the element referred to can be attachable directly to another article or interconnected by attachment to an intermediate member that attaches to the article.

In other embodiments, operational methods that enable tilting of a display screen of a diagnostic analyzer are provided. Accordingly, the inventors have invented new apparatus and methods that enable easy tilt angle adjustment of a display screen of a diagnostic analyzer in order to clear a potential interference with another item, such as a storage cabinet or cabinet door, and/or removal of a hand-held device (e.g., a tablet) containing the display screen therefrom, so that point-of-care scanning of patient or other information can be accomplished.

In one aspect of the disclosure, a tiltable display screen assembly is provided. The tiltable display screen assembly comprises a tiltable support (which may be made up of one or more pieces) configured to be interconnectable to a hand-held device comprising a display screen, a multi-angle adjuster having multiple adjustment features, and a tilting screen mechanism that is pivotable relative to the tiltable support. The tilting screen mechanism can further comprise a shaft, one or more moveable paddles coupled to the shaft, and an adjustment member coupled to the shaft (e.g., connected to and extending radially from the shaft) and having an end configured to engage with two or more of the multiple adjustment features of the multi-angle adjuster in order to adjust a tilt angle of the tiltable support.

In another aspect of the disclosure, a diagnostic analyzer is provided. The diagnostic analyzer, which can be a blood gas analyzer or other bio-liquid diagnostic analyzer, comprises a base and a hand-held device rotatably coupled to the base. The hand-held device comprises a display screen. The diagnostic analyzer further comprises a tiltable display screen assembly interconnected between the base and the hand-held device. The tiltable display screen assembly comprises a tiltable support interconnected to the hand-held device, the tiltable support comprising a support bracket attached to a mounting member configured to attach to the hand-held device, a multi-angle adjuster coupled to the base, the multi-angle adjuster having multiple adjustment features thereon, and a tilting screen mechanism pivotable relative to the tiltable support. The tilting screen mechanism can comprise a shaft, one or more moveable paddles coupled to the shaft, and an adjustment member depending from the shaft and having an end configured to engage with two or more of the multiple adjustment features of the multi-angle adjuster in order to adjust a tilt of the display screen relative to the base.

In another aspect of the disclosure, a method of positioning a display screen of a diagnostic analyzer is provided. The method comprises providing a base of the diagnostic analyzer, providing a hand-held device comprising a display screen, and interconnecting a tiltable display screen assembly between the base and the hand-held device, wherein the tiltable display screen assembly comprises a tiltable support. The tiltable support comprises a support bracket attached to a mounting member that is attached to the hand-held device, a multi-angle adjuster coupled to the base, the multi-angle adjuster having multiple adjustment features thereon, and a tilting screen mechanism pivotable relative to the tiltable support. The tilting screen mechanism comprises a shaft, one or more moveable paddles coupled to the shaft, and an adjustment member depending from the shaft and having an end configured to engage with at least some of the multiple adjustment features. The method further comprises depressing the one or more paddles to disengage the end of the adjustment member from one of the multiple adjustment features in order to tilt the tiltable support relative to the base and adjust a tilt angle (TA) of the display screen.

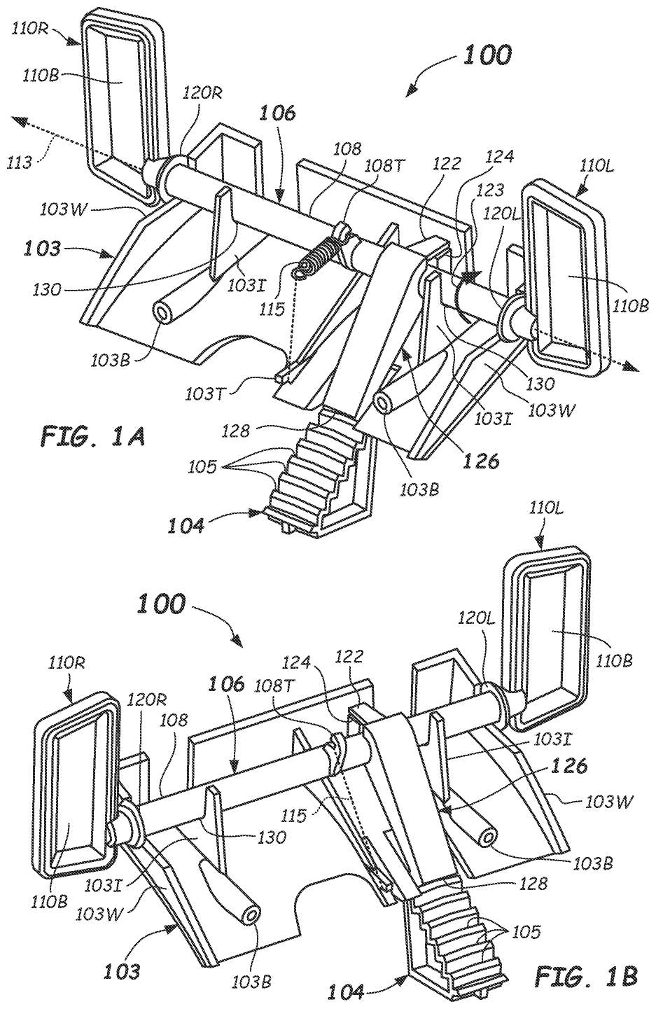

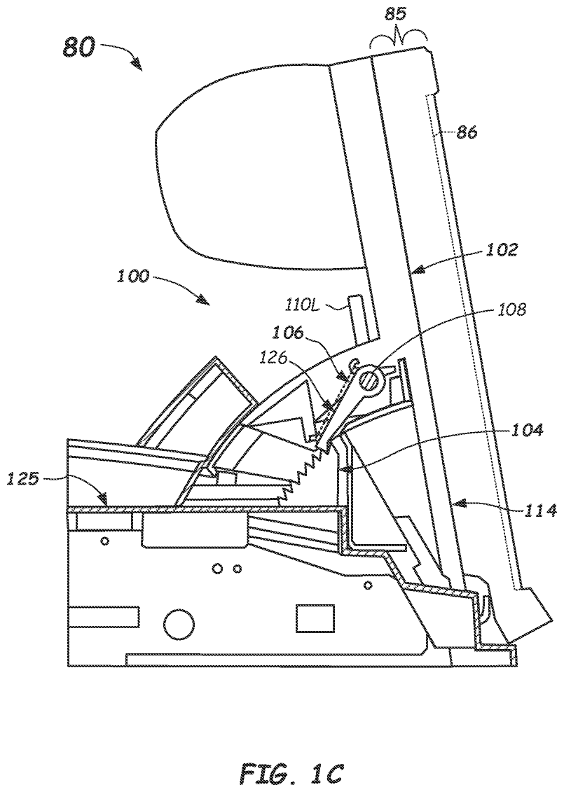

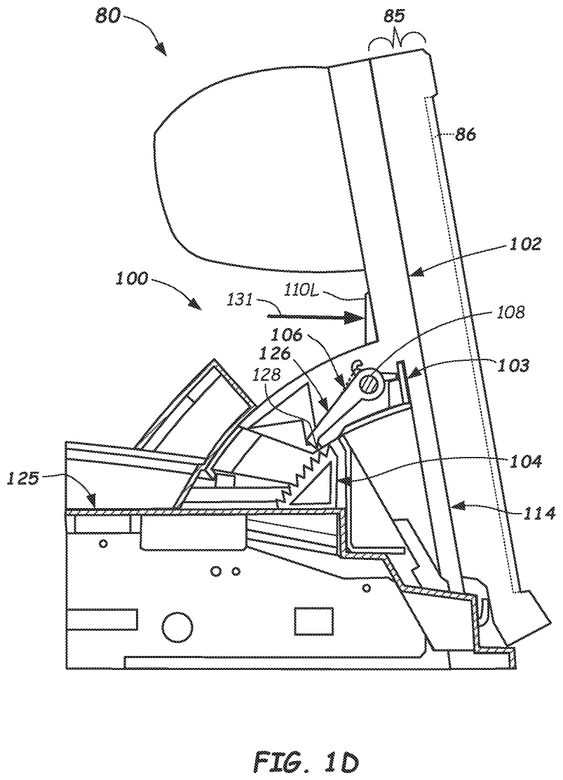

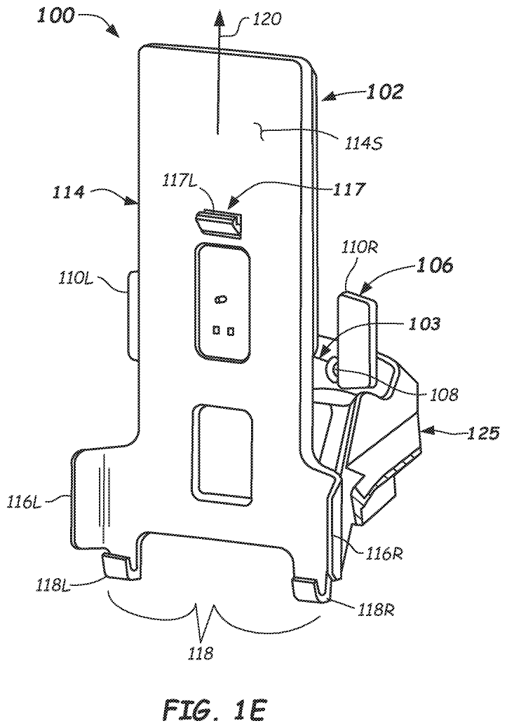

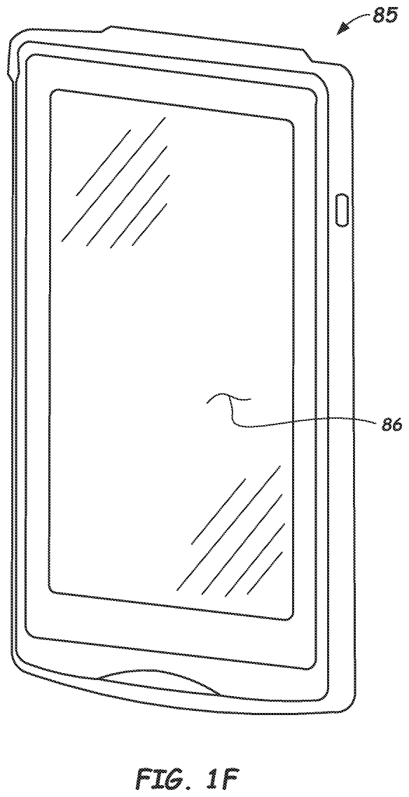

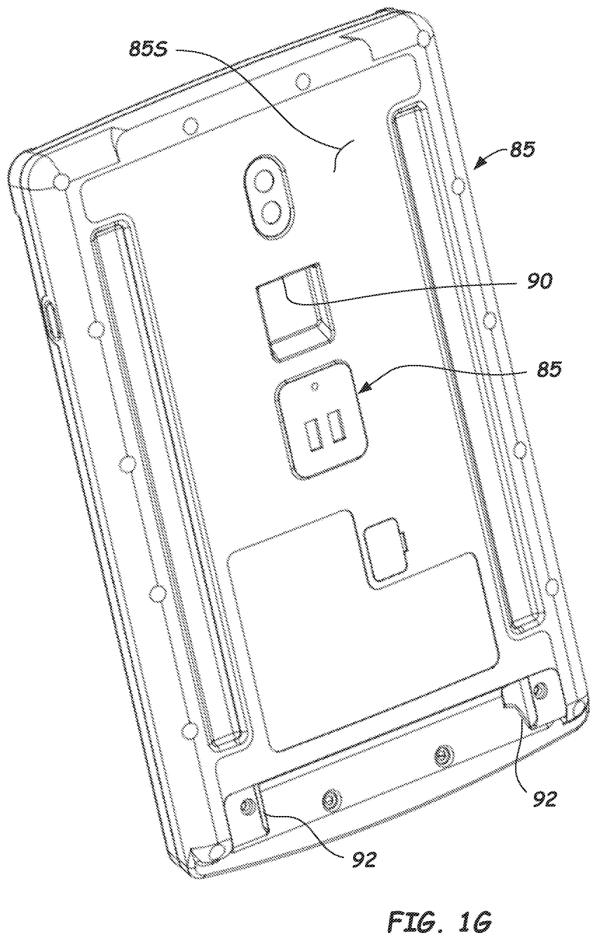

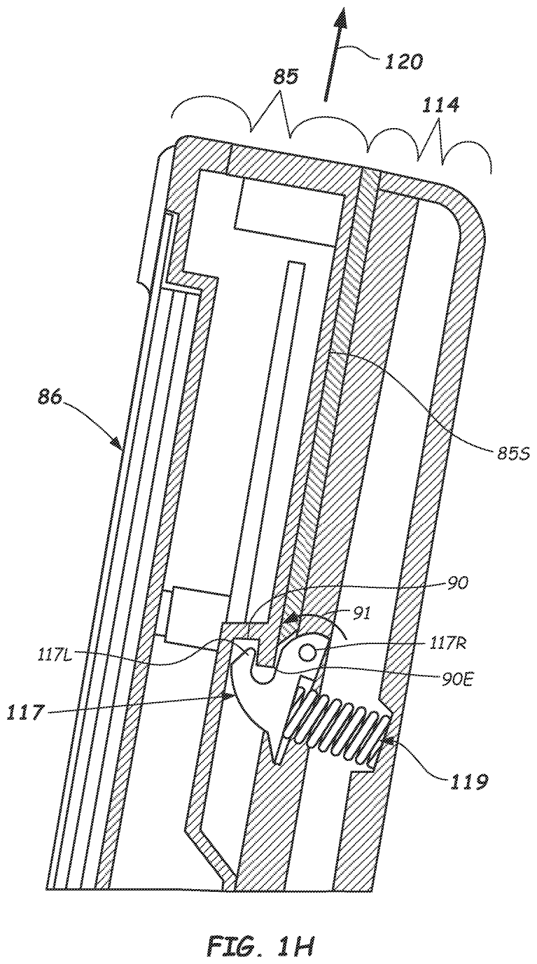

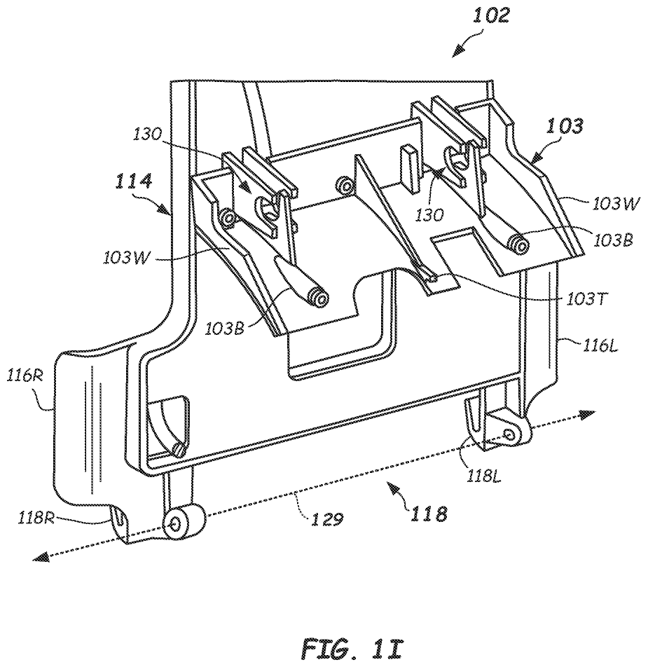

In accordance with the disclosure, several example embodiments of apparatus and methods configured to enable tilt angle adjustment of a display screen of a diagnostic analyzer and/or removal of a hand-held device comprising the display screen from a diagnostic analyzer are shown and described with reference to FIGS. 1A-3 hereof.

FIGS. 1A-1E and 1H-1I illustrate a first example embodiment of a tiltable display screen assembly 100, which can be an integrated part of a diagnostic analyzer 80 (FIGS. 1C-1D) and is operable to allow adjustment of a tilt angle (TA) of a hand-held device 85 (FIG. 1F-1G) thereof, which can be a removable part of the diagnostic analyzer 80 in some embodiments. For example, the hand-held device 85 can be a removable from the tiltable display screen assembly 100 in order to accomplish any number of point-of-care functions, such as to scan with a bar code reader integral with the hand-held device 85 or otherwise obtain with the hand-held device 85, patient information, medication information, and/or other diagnostic or medical condition information. The details of the rest of the diagnostic analyzer 80 other than the tiltable feature are not described herein and are conventional.

The tiltable display screen assembly 100 can be used with any type of diagnostic analyzer 80 where a biological sample is desired to be introduced and tested within the diagnostic analyzer 80. For example, the tiltable display screen assembly 100 is very useful for point-of-care diagnostic analyzers, such as blood gas analyzers, and the like. In particular, the tiltable display screen assembly 100 can be used to allow the display screen 86 to be rotated so that the overall height of the diagnostic analyzer 80 can be lowered. Through such lowering, the diagnostic analyzer 80 may fit underneath of an impediment such as a storage cabinet or otherwise lowered to avoid interference with operation of a cabinet door of the storage cabinet. The ability to move the diagnostic analyzer 80 underneath of the storage cabinet may free up counter space to use for other purposes.

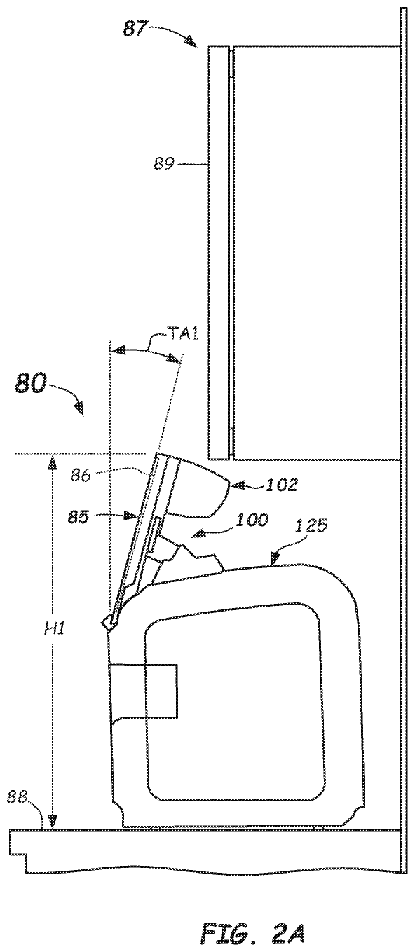

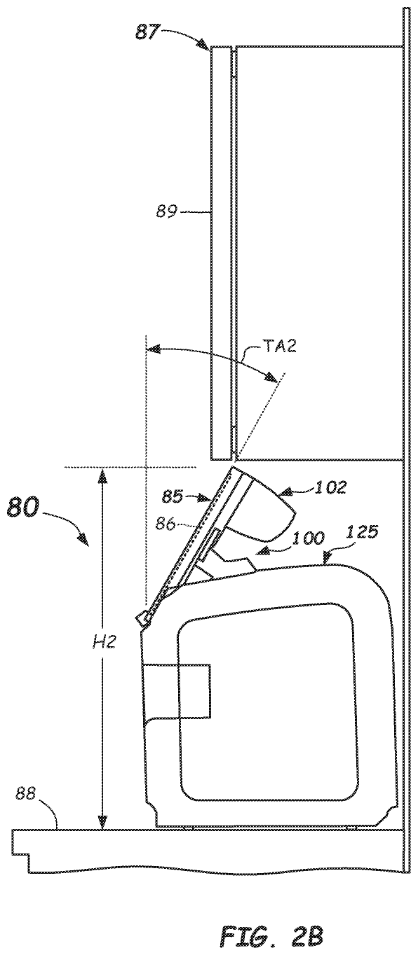

As best shown in FIGS. 2A and 2B, the diagnostic analyzer 80 is shown having a base 125, that may comprise a housing structure containing some or all of the diagnostic components of the diagnostic analyzer 80. FIGS. 2A and 2B also illustrate the hand-held device 85 mounted to the tiltable support 102. For example, FIG. 2A illustrates the hand-held device 85 including the display screen 86 shown in an upright but slightly tilted position with a tilt angle TA1 from the vertical of about 10 degrees. This tilt angle TA1 may enable ease of view of the display screen 86 by medical personnel. However, the overall height H1 of the diagnostic analyzer 80 may be too tall in some cases to be slid under a storage cabinet 87 located above a countertop surface 88 on which the diagnostic analyzer 80 sits. In some instances, the Height H1 may also block the cabinet door 89 of the storage cabinet 87 from being opened sufficiently so that various supplies can be accessed.

The tiltable display screen assembly 100, to which the hand-held device 85 is interconnected, allows the tiltable support 102 and thus the interconnected hand-held device 85 containing the display screen 86 to be tilted to a lower height H2 as shown in FIG. 2B. In one example, this allows the cabinet door 89 to be opened and clear the top of the hand-held device 85 without moving the base 125 of the diagnostic analyzer 80. In another example, the tiltable display screen assembly 100 allows the tiltable support 102 and thus the interconnected hand-held device 85 containing the display screen 86 to be tilted to a lower height H2 so that the diagnostic analyzer 80 can be slid underneath of the storage cabinet 87 as shown in FIG. 2B and free up more countertop space.

In more detail, the tiltable display screen assembly 100 comprises a tiltable support 102, a multi-angle adjuster 104, and a tilting screen mechanism 106. The tiltable support 102 is configured to be interconnectable to a hand-held device 85 comprising the display screen 86. Tiltable support 102 (FIGS. 1E and 1I) can comprise a mounting member 114 in some embodiments that is configured to attach to the hand-held device 85 comprising the display screen 86. The tiltable support 102 may, in some embodiments, be a single piece, or, as in the first embodiment, made up of two interconnected pieces.

For example, the tiltable support 102 can be made up of the support bracket 103 as shown in FIGS. 1A-1B and a mounting member 114 as shown in FIGS. 1E and 1I. The hand-held device 85 can be any computing article or device that can be handled by being hand held by medical personnel and that includes a processor and display screen 86, such as a computer tablet, mobile phone, or the like. The display screen 86 can be a LCD, LED, OLED, and/or AMOLED, and may have a screen resolution of 800×600 dpi or higher, for example. The display screen 86 may be fixed to the body of the hand-held device 85. Other suitable screen types may be used, as well as other screen resolutions.

The support bracket 103, as best shown in FIGS. 1A, 1B, and 1I comprises a formed body, made from an injection-molded plastic, for example. The body may include sidewalls 103W, which may be vertically oriented and spaced apart. The body may further include a spring mounting tab 103T to be described later herein. One or more connection bosses 103B or holes may be included that receive fasteners (FIG. 1I) to attach the support bracket 103 to the backside of the mounting member 114. The mounting member 114 can be configured to be detachably mounted to the hand-held device 85 comprising the display screen 86.

Mounting member 114 can include a device support surface 114S configured to contact a back surface 85S of the hand-held device 85 as shown in FIGS. 1C, 1D, 1E, and 1G. The mounting member 114 may include side retaining features 116L, 116R (shown in FIGS. 1E and 1I), such as walls or other suitable location fixing or restraining features that may engage with and/or otherwise restrain lateral (horizontal as shown) motion of the hand-held device 85 on the mounting member 114. In some embodiments, a slight gap may be allowed between the sides of the hand-held device 85 and the side retaining features 116L, 116R allowing only limited lateral motion. Mounting member 114 can, in some embodiments, include lower support 118 that can comprise one or more support hooks. In the depicted embodiment, multiple ones of the support hooks 118L, 118R are spaced apart as shown in FIGS. 1E and 1I. The one or more support hooks 118L, 118R can be configured to receive, contact, and support a bottom of the hand-held device 85. In some embodiments, the lower support 118 may engage with adapters 92, such as hooks, mounted on, or integral with, the backside of the hand-held device 85 as is shown in FIG. 1G. Other suitable structural configurations of the lower support 118 may be used to support the hand-held device 85.

In some embodiments, mounting member 114 can include a latch 117. Latch 117 may be a spring-loaded protrusion that is configured to be received in a catch feature 90 formed, for example, as a recess in a back surface 85S of the hand-held device 85 as shown in FIGS. 1E, 1G, and 1H. In particular, latch 117 can be received and capturable in the catch feature 90 of the hand-held device 85, such as in an undercut pocket shown formed on the back surface 85S of the hand-held device 85.

Once mounted to the mounting member 114, the hand-held device 85 can be removed from the mounting member 114 by pulling up on the hand-held device 85 in the direction of arrow 120 (FIGS. 1E and 1H). After using the hand-held device 85, such as to obtain (scan) patient bar code information and/or to obtain other pertinent information, the hand-held device 85 can be remounted to the mounting member 114 by inserting the adapters 92 at the bottom of the hand-held device 85 into the lower support 118 (e.g., into the one or more of the support hooks 118L, 118R) and rotating the top of the hand-held device 85 towards the latch 117. As the latch 117 is contacted by the edge 90E (FIG. 1H) of the catch feature 90, the latch 117 is rotated downward (e.g., counter clockwise as indicated by arrow 91) so that the locking tab 117L can rotate about the axis of rotation 117R and pass by the edge 90E of the catch feature 90 and then rotate (clockwise, opposite direction of arrow 91) back into the catch feature 90 (e.g., the undercut pocket) as shown in FIG. 1H.

Thus, FIG. 1H illustrates that the latch 117 has been rotated clockwise (opposite of arrow 91) about axis of rotation 117R under a restorative force of a latch spring 119 and engaged in the catch feature 90 (undercut pocket). Other suitable latch and catch mechanisms may be used to secure the hand-held device 85 to the mounting member 114, as well as other spring types.

Again referring to FIGS. 1A-1D, the tiltable display screen assembly 100 comprises a multi-angle adjuster 104 having multiple adjustment features 105. The multi-angle adjuster 104 may be coupled to a base 125 of a diagnostic analyzer 80, and the multiple adjustment features 105 may include two or more of the multiple adjustment features 105. The multiple adjustment features 105 may be steps, ribs, or other suitable engagement features configured to engage with end 128 to allow adjustment of the tilt angle (TA) of the tiltable support 102. The coupling to the base 125 may be by any suitable fastening means, such as by using mechanical fastening (e.g., one or more screws), capturing between pieces of the base 125, using snap-fit features in order to snap fit to the base 125, making the multi-angle adjuster 104 integral with the base 125, gluing to the base 125 by using adhesive, or otherwise bonding to the base, and the like. The multiple adjustment features 105 comprising two or more steps can provide for two or more tilt angle (TA) adjustment increments. However, in some embodiments, there may be five or more tilt angle adjustment increments, such as by implementing five or more steps. Seven steps are shown in the depicted embodiment, allowing seven discreet tilt angle (TA) adjustments of the display screen 86 relative to the base 125. Other numbers of discreet tilt angle (TA) adjustments greater than two discreet tilt angle (TA) adjustments may be used.

The tiltable display screen assembly 100 further comprises a tilting screen mechanism 106 that is operationally pivotable relative to the tiltable support 102, such as relative to the support bracket 103, such as when the tiltable support 102 comprises a two-piece construction comprising support bracket 103 and mounting member 114. However, in some embodiments, the mounting member 114 and support bracket 103 may be molded as one piece. In either configuration, the tiltable support 102 comprises a mounting member 114, and the hand-held device 85 can be readily detachable from the mounting member 114.

Tilting screen mechanism 106 can further comprise a shaft 108, one or more moveable paddles 110R, 110L (FIGS. 1A, 1B, 1E), and an adjustment member 126. The tiltable support 102 can comprises a support bracket 103 to which the shaft 108 is pivotally mounted. The shaft 108 can have any desired shape to accommodate the pivoting (rotation) about its rotational axis 113 (FIG. 1A). In particular, shaft 108 may be supported by and ride (rotate) in one or more bearing journals, such as bearing supports 130 at least partially formed from the support bracket 103, which can be spaced apart formed-in features of the support bracket 103, such as at least partially formed on internal walls 1031. Bearing supports 130 operate to support the shaft 108 and sufficiently limit radial motion thereof so that the shaft 108 may freely rotate, yet is supported to restrain its radial position to substantially form the rotational axis 113 that is radially fixed. Bearing supports 130 may include slight amount of slop relative to the diameter of the shaft 108 at the bearing supports 130 to allow such free rotation of the shaft 108.

As best shown in FIG. 1I, bearing supports 130 can be formed from features formed on the back surface of the mounting member 114 and formed-in features of the support bracket 103 that interact with each other to form bearing journals that capture the shaft 108 of the tilting screen mechanism 106. Optionally, bearing supports 130 may be formed in sidewalls 103W and the shaft 108 or otherwise formed by any suitable means.

The one or more moveable paddles, such as moveable paddles 110R, 110L (FIGS. 1A, 1B, 1E), may be coupled to one or more outer ends of the shaft 108. In some embodiments, the one or more moveable paddles 110L, 110R can have a first one of the one or more moveable paddles 110L, 110R attached to a first end of the shaft 108 and second one of the one or more moveable paddles 110L, 110R attached to a second opposite end of the shaft 108, for example. Further, the shaft 108 can include axial restraining features 120L, 120R (FIGS. 1A, 1B) that operate to limit axial motion of the shaft 108 relative to the tiltable support 102, and in particular relative to the support bracket 103. In some embodiments, the axial restraining features 120L, 120R can have an annular shape and may be positioned outside of the support walls 130W. Axial restraining features 120L, 120R may be integrally molded on the shaft 108, for example.

Furthermore, a stop appendage 122 may depend from the shaft 108 and can have an end portion that is configured to engage with a limit stop 124 in order to limit rotation of the shaft 108 relative to the tiltable support 102 within limits. For example, contact between the underside of stop appendage 122 and the top of the limit stop 124 as shown in FIGS. 1A and 1B may allow limited rotation in the direction of arrow 123. Rotation may be limited to between enough rotation to have the end 128 clear the multiple adjustment features 105 (e.g., the steps) and about 20 degrees, for example.

The one or more moveable paddles 110R, 110L may be elements having one or more surfaces that are generally co-parallel with a surface of the display screen 86. To aid with tactile feel, a backside of the one or more moveable paddles 110L, 110R may include a cutout or depressed back 110B, with an ideal configuration including a recessed portion that may be large enough to accommodate at least two fingertips of the medical personnel operating the tilting screen mechanism 106. This allows for uncoupling of the tilting screen mechanism 106 by depressing of one or more of the moveable paddles 110L, 110R via pinching between the front of the hand-held device with the thumb and contacting the backside of the one or more paddles 110L, 110R with one or more fingers of the medical personnel.

Tilting screen mechanism 106 can further comprise an adjustment member 126 depending from the shaft 108 and having an end 128 that is configured to engage with two or more of the multiple adjustment features 105 of the multi-angle adjuster 104 in order to adjust a tilt of the tiltable support 102 and thus tilt angle (TA) of the hand-held device 85 and the display screen 86 thereof. When the one or more moveable paddles 110L, 110R are depressed in the direction of arrow 131 in FIG. 1D, the shaft 108 is rotated, and the end 128 of the adjustment member 126 is disengaged from one of the multiple adjustment features 105 of the multi-angle adjuster 104. The tiltable support 102 can then be rotated (pivoted) about a mount axis 129 (FIG. 1I) to another tilt angle (such as TA2 shown in FIG. 2B) and the one or more moveable paddles 110L, 110R can then be released to again engage the end 128 with a different one of the multiple adjustment features 105.

Mount axis 129 is an axis of rotation of the mounting member 114 relative to the base 125 of the diagnostic analyzer 80 and can be formed by any suitable structure, such as an axle or pins interfacing with portions of the bottom of the mounting member 114 and front of the base 125, for example. The tiltable support 102 may be adjustable over a tilt angle (TA) of at least 15 degrees, or even at least 20 degrees, for example. In some embodiments, tilt angle (TA) may be adjustable between about 5 degrees and 45 degrees, or even between about 10 degrees and 35 degrees, for example.

The shaft 108 may have a spring 115 attached thereto that is offset a radial distance from a rotational axis 113 thereof. For example, the spring 115 as shown in FIG. 1A (shown as a dotted line in FIGS. 1B-1D) may be attached to a spring mounting tab 108T of the shaft 108 that is radially offset from the rotational axis 113 of the shaft 108. The offset may be about 0.25 inches and the spring rate may be between 1 and 2 pounds/inch, for example. Other suitable offsets and spring rates, as well as spring types other than coil springs may be used. In some embodiments, the spring 115 is further attached to a support bracket 103 of the tiltable support 102. Thus, the spring 115 can be attached and secured between spring mounting tab 103T of the support bracket 103 and spring mounting tab 108T of the shaft 108. This spring 115 provides a spring bias of the tilting screen mechanism 106 to a normally engaged position, wherein the end 128 of the adjustment member 126 is normally engaged with one of the multiple adjustment features 105 (e.g., steps or the like). For clarity and illustration purposes, the spring 115 is shown detached in FIG. 1A, but is attached in use.

Thus, in some embodiments, a diagnostic analyzer 80 is provided that comprises a base 125, a hand-held device 85 having a display screen 86 rotatably (i.e., pivotally) coupled to the base 125, and a tiltable display screen assembly 100 as described herein interconnected between the base 125 and the hand-held device 85. As discussed above, the tiltable display screen assembly 100 can comprise a tiltable support 102 interconnected to the hand-held device 85, a multi-angle adjuster 104, and a tilting screen mechanism 106. The tiltable support 102 can comprise a support bracket 103 attached to a mounting member 114 that configured to attach to the hand-held device 85. The multi-angle adjuster 104 can be directly or indirectly coupled to the base 125 of the diagnostic analyzer 80. The multi-angle adjuster 104 can have multiple adjustment features 105 formed thereon, such as two or more adjustment features 105. The tilting screen mechanism 106 can be pivotable relative to the tiltable support 102, and can comprise a shaft 108, one or more moveable paddles 110L, 110R coupled to the shaft 108, and an adjustment member 126 depending from the shaft 108 and having an end 128 configured to engage with two or more of the multiple adjustment features 105 of the multi-angle adjuster 104 in order to adjust a tilt of the display screen 86 relative to the base 125.

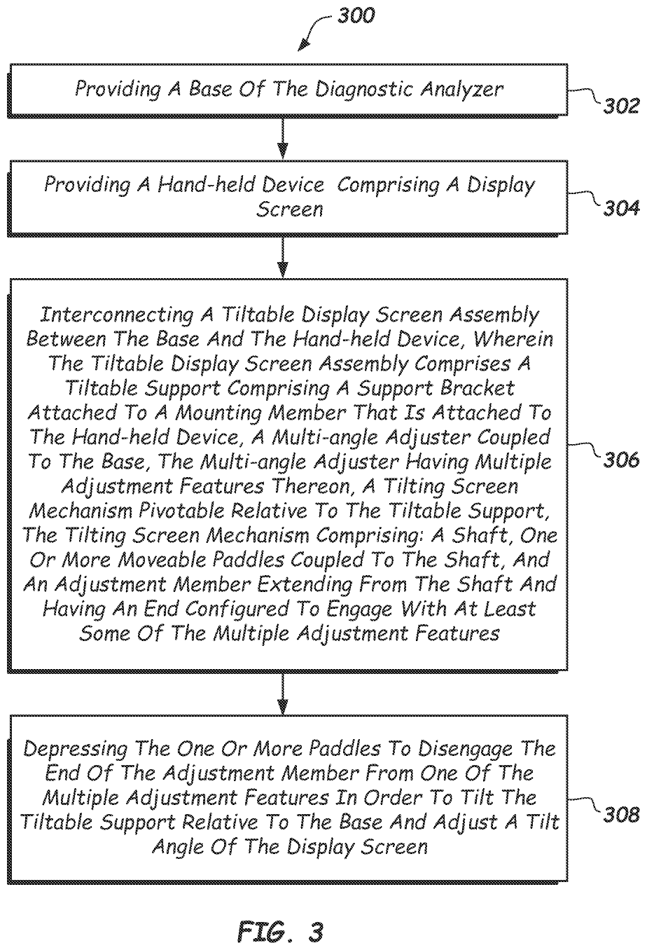

Referring now to FIG. 3, and in another aspect, a method 300 of positioning a display screen 86 of a diagnostic analyzer 80 is provided. The method 300 comprises, in block 302, providing a base 125 of the diagnostic analyzer 80, and, in block 304, providing a hand-held device 85 comprising a display screen 86.

The method 300 further comprises, in block 306, interconnecting a tiltable display screen assembly 100 between the base 125 and the hand-held device 85, wherein the tiltable display screen assembly 100 comprises: a tiltable support 102 comprising a support bracket 103 attached to a mounting member 114 that is attached to the hand-held device 85, a multi-angle adjuster 104 coupled to the base 125, the multi-angle adjuster 104 having multiple adjustment features 105 thereon, and a tilting screen mechanism 106 pivotable relative to the tiltable support 102. The tilting screen mechanism 106 comprises a shaft 108, one or more moveable paddles 110L, 110R coupled to the shaft 108, and an adjustment member 126 depending from the shaft 108 and having an end 128 configured to engage with at least some of the multiple adjustment features 105.

The method 300 further includes, in block 308, depressing the one or more moveable paddles 110L, 110R to disengage the end 128 of the adjustment member 126 from one of the multiple adjustment features 105 in order to tilt the tiltable support 102 relative to the base 125 and adjust a tilt angle (TA) of the display screen 86.

As should be recognized, the various sequences involved in the method 300 may take place in any desired order.

The following is a list of non-limiting illustrative embodiments disclosed herein:

1. A tiltable display screen assembly of a first illustrative embodiment, comprising:
a tiltable support configured to be interconnectable to a hand-held device comprising a display screen;
a multi-angle adjuster having multiple adjustment features; and
a tilting screen mechanism pivotable relative to the tiltable support, the tilting screen mechanism comprising:
   a shaft,
   one or more moveable paddles coupled to the shaft, and
   an adjustment member extending from the shaft and having an end configured to engage with two or more of the multiple adjustment features of the multi-angle adjuster in order to adjust a tilt angle of the tiltable support.

2. The tiltable display screen assembly of the first illustrative embodiment, wherein the tiltable support comprises a support bracket, and wherein the shaft is pivotally mounted to the support bracket.

3. The tiltable display screen assembly of any one of the illustrative embodiments 1 to 2, wherein the support bracket is attached to a mounting member configured to attach the hand-held device comprising the display screen.

4. The tiltable display screen assembly of any one of the illustrative embodiments 1 to 3, wherein the tiltable support comprises a mounting member configured to be attachable to the hand-held device comprising the display screen.

5. The tiltable display screen assembly of any one of the illustrative embodiments 1 to 4, wherein the mounting member comprises one or more support hooks configured to receive a bottom of the hand-held device.

6. The tiltable display screen assembly of any one of the illustrative embodiments 1 to 5, wherein the mounting member comprises a latch configured to be received in a catch feature on a back surface of the hand-held device.

7. The tiltable display screen assembly of any one of the illustrative embodiments 1 to 6, wherein the tiltable support comprises support bracket and a mounting member.

8. The tiltable display screen assembly of any one of the illustrative embodiments 1 to 7, wherein the one or more moveable paddles comprise a first moveable paddle attached to a first end of the shaft and second moveable paddle attached to a second end of the shaft.

9. The tiltable display screen assembly of any one of the illustrative embodiments 1 to 8, further comprising a spring attached to the shaft, wherein the spring is attached offset from a rotational axis of the shaft.

10. The tiltable display screen assembly of any one of the illustrative embodiments 1 to 9, wherein the spring is further attached to a support bracket of the tiltable support.

11. The tiltable display screen assembly of any one of the illustrative embodiments 1 to 10, wherein the shaft has axial restraining features that limit axial motion of the shaft relative to the tiltable support.

12. The tiltable display screen assembly of any one of the illustrative embodiments 1 to 11, wherein the shaft is supported and radially restrained in bearing supports of a support bracket.

13. The tiltable display screen assembly of any one of the illustrative embodiments 1 to 12, comprising a stop appendage extending from the shaft and having an end portion configured to engage with a limit stop to limit rotation of the shaft relative to the tiltable support.

14. The tiltable display screen assembly of any one of the illustrative embodiments 1 to 13, wherein the multi-angle adjuster is coupled to a base, and the multi-angle adjuster comprises two or more steps.

15. The tiltable display screen assembly of any one of the illustrative embodiments 1 to 14, wherein the tiltable support is adjustable over a tilt angle of at least 15 degrees.

16. The tiltable display screen assembly of any one of the illustrative embodiments 1 to 15, wherein the tiltable support comprises a mounting member and the hand-held device is detachable from the mounting member.

17. The tiltable display screen assembly of any one of the illustrative embodiments 1 to 16, wherein the mounting member comprises one or more support hooks configured to receive a bottom of the hand-held device, and a spring-loaded latch configured to be received in a catch feature on a back surface of the hand-held device.

18. The tiltable display screen assembly of any one of the illustrative embodiments 1 to 17, wherein the mounting member comprises a mount axis, and the mounting member is tiltable about the mount axis.

19. A diagnostic analyzer, comprising:
a base;
a hand-held device rotatably coupled to the base; and
a tiltable display screen assembly interconnected between the base and the hand-held device, the tiltable display screen assembly comprising any one of the illustrative embodiments 1 to 18.

20. A method of positioning a display screen of a diagnostic analyzer, comprising:
providing a base of the diagnostic analyzer;
providing a hand-held device comprising the display screen;
interconnecting a tiltable display screen assembly between the base and the hand-held device, wherein the tiltable display screen assembly comprises any one of the illustrative embodiments 1 to 18; and
depressing the one or more moveable paddles to disengage the end of the adjustment member from one of the multiple adjustment features in order to tilt the tiltable support relative to the base and adjust a tilt angle of the display screen.

While this disclosure is susceptible to various modifications and alternative forms, specific assembly, apparatus, diagnostic analyzer, and method embodiments thereof have been shown by way of example in the drawings and are described in detail herein. It should be understood, however, that the particular assembly, apparatus, diagnostic analyzer, and methods disclosed herein are not intended to limit the disclosure or the claims.

What is claimed is:

1. A tiltable display screen assembly, comprising:
a tiltable support configured to be interconnectable to a display screen;
a multi-angle adjuster having multiple adjustment features; and
a tilting screen mechanism pivotable relative to the tiltable support, the tilting screen mechanism comprising:
a shaft,
one or more moveable paddles coupled to the shaft, and
an adjustment member extending from the shaft and having an end configured to engage with two or more of the multiple adjustment features of the multi-angle adjuster in order to adjust a tilt angle of the tiltable support.

2. The tiltable display screen assembly of claim 1, wherein the tiltable support comprises a support bracket, and wherein the shaft is pivotally mounted to the support bracket.

3. The tiltable display screen assembly of claim 2, wherein the support bracket is attached to a mounting member configured to attach to the display screen.

4. The tiltable display screen assembly of claim 1, wherein the tiltable support comprises a mounting member configured to be attachable to a hand-held device comprising the display screen.

5. The tiltable display screen assembly of claim 4, wherein the mounting member comprises one or more support hooks configured to receive a bottom of the hand-held device.

6. The tiltable display screen assembly of claim 4, wherein the mounting member comprises a latch configured to be received in a catch feature on a back surface of the hand-held device.

7. The tiltable display screen assembly of claim 1, wherein the tiltable support comprises a support bracket and a mounting member.

8. The tiltable display screen assembly of claim 1, wherein the one or more moveable paddles comprise a first moveable paddle attached to a first end of the shaft and a second moveable paddle attached to a second end of the shaft.

9. The tiltable display screen assembly of claim 1, further comprising a spring attached to the shaft, wherein the spring is attached offset from a rotational axis of the shaft.

10. The tiltable display screen assembly of claim 9, wherein the spring is further attached to a support bracket of the tiltable support.

11. The tiltable display screen assembly of claim 1, wherein the shaft has axial restraining features that limit axial motion of the shaft relative to the tiltable support.

12. The tiltable display screen assembly of claim 1, wherein the shaft is supported and radially restrained in bearing supports of a support bracket.

13. The tiltable display screen assembly of claim 1, comprising a stop appendage extending from the shaft and having an end portion configured to engage with a limit stop to limit rotation of the shaft relative to the tiltable support.

14. The tiltable display screen assembly of claim 1, wherein the multi-angle adjuster is coupled to a base, and the multi-angle adjuster comprises two or more steps.

15. The tiltable display screen assembly of claim 1, wherein the tiltable support is adjustable over a tilt angle of at least 15 degrees.

16. The tiltable display screen assembly of claim 1, wherein the tiltable support comprises a mounting member and the display screen is detachable from the mounting member.

17. The tiltable display screen assembly of claim 16, wherein the mounting member comprises one or more support hooks configured to receive a bottom of the display screen, and a spring-loaded latch configured to be received in a catch feature on a back surface of the display screen.

18. The tiltable display screen assembly of claim 16, wherein the mounting member comprises a mount axis, and the mounting member is tiltable about the mount axis.

19. A diagnostic analyzer, comprising:
a base;
a display screen rotatably coupled to the base; and
a tiltable display screen assembly interconnected between the base and the display screen, the tiltable display screen assembly comprising:
a tiltable support interconnected to the display screen, the tiltable support comprising a support bracket attached to a mounting member configured to attach to the display screen;
a multi-angle adjuster coupled to the base, the multi-angle adjuster having multiple adjustment features thereon; and
a tilting screen mechanism pivotable relative to the tiltable support, the tilting screen mechanism comprising:
a shaft,
one or more moveable paddles coupled to the shaft, and
an adjustment member extending from the shaft and having an end configured to engage with two or more of the multiple adjustment features of the multi-angle adjuster in order to adjust a tilt angle of the display screen relative to the base.

20. A method of positioning a display screen of a diagnostic analyzer, comprising:
providing a base of the diagnostic analyzer;
providing a display screen;
interconnecting a tiltable display screen assembly between the base and the display screen, wherein the tiltable display screen assembly comprises a tiltable support comprising a support bracket attached to a mounting member that is attached to the display screen, a multi-angle adjuster coupled to the base, the multi-angle adjuster having multiple adjustment features thereon, a tilting screen mechanism pivotable relative to the tiltable support, the tilting screen mechanism comprising: a shaft, one or more moveable paddles coupled to the shaft, and an adjustment member extending from the shaft and having an end configured to engage with at least some of the multiple adjustment features; and
depressing the one or more moveable paddles to disengage the end of the adjustment member from one of the multiple adjustment features in order to tilt the tiltable support relative to the base and adjust a tilt angle of the display screen.
